(19) United States
(12) Patent Application Publication
(10) Pub. No.: US 2026/0118236 A1
(43) Pub. Date: Apr. 30, 2026

(54) MOLD, SYSTEM FOR TESTING SAMPLE, METHOD OF TESTING SAMPLE AND SAMPLE HAVING COMPRESSIBLE BODY

(71) Applicant: ASAHI INTECC CO., LTD., Seto-shi (JP)

(72) Inventor: Hirobumi WATANABE, Irvine, CA (US)

(73) Assignee: ASAHI INTECC CO., LTD., Seto-shi (JP)

(21) Appl. No.: 19/426,201

(22) Filed: Dec. 19, 2025

Related U.S. Application Data
(63) Continuation of application No. PCT/JP2024/023027, filed on Jun. 25, 2024.
(60) Provisional application No. 63/514,708, filed on Jul. 20, 2023.

(51) Int. Cl. G01N 3/04; G01N 33/483

(52) U.S. Cl. CPC G01N 3/04; G01N 33/4833; G01N 2203/0252

(57) ABSTRACT

A mold can include two chucks and two or more central mold parts that are configured to be removably coupled to the chucks such that the central mold parts extend between the chucks. When the central mold parts are coupled to the chucks, the central mold parts and the chucks can define a mold cavity including a gauge-forming portion and two shoulder-forming portions. The gauge-forming portion can be defined by the central mold parts and can have a first transverse dimension. The shoulder-forming portions can each be defined by a respective one of the chucks and have a second transverse dimension that is at least 1.5 times the first transverse dimension.

### CROSS REFERENCE TO RELATED APPLICATIONS

The present application is a bypass continuation of International Application No. PCT/JP2024/023027 filed on Jun. 25, 2024, which claims priority to U.S. Provisional Application 63/514,708 filed Jul. 20, 2023, the entire contents of each of which being incorporated herein by reference.

### TECHNICAL FIELD

The present disclosure relates generally to tensile testing of molded samples.

### BACKGROUND ART

Tensile testing of materials can be used to ascertain one or more mechanical properties of the material. For example, when a sample is pulled apart, the tensile force exerted on the sample and the deformation of the sample (e.g., its change in length) can be measured. From these measurements, the stress/strain relationship for the sample can be determined and a variety of mechanical properties such as yield strength, ultimate tensile strength, and modulus of elasticity can be calculated.

To promote reliability and consistency in tensile testing across different samples, the samples can be shaped to encourage failure in the same portion of the samples. Some tensile tests, like those in ASTM D638 employing the Type IV Test Specimen, involve machining a sample into a “dogbone” shape having a smaller-diameter gauge portion disposed between two larger-diameter shoulder portions. The shoulder portions can provide a convenient location for gripping the sample during the tensile test, and the stress in the shoulder portions during a tensile test can be lower than that in the gauge portion because the shoulder portions have a larger diameter than the gauge portion, which in turn can encourage failure in the gauge portion rather than in the shoulder portions.

Tensile testing of compressible materials can face challenges in reliability and consistency even if the sample is machined to have the dogbone shape. In tensile tests, the sample is typically held by two grips that each clamp a respective end of the sample and are pulled apart to exert a tensile force on the sample. The clamping of the ends of a compressible sample can cause deformation and induce stress concentrations in the sample's ends and can accordingly increase the risk of unintentional failure in the ends of the sample. For example, when testing a dogbone-shaped, compressible sample, sample failure may unintentionally occur in one of the shoulder portions rather than in the gauge portion of the sample, which can decrease the reliability and consistency of the tensile test.

### SUMMARY

The present molds, systems, and methods can be used to better promote reliability and consistency in the tensile testing of compressible samples. To do so, the mold can include two chucks and two or more central mold parts that are configured to be removably coupled to the chucks such that the central mold parts extend between the chucks. When the central mold parts are coupled to the chucks, the chucks and the central mold parts can define a mold cavity having a gauge-forming portion and two shoulder-forming portions. The gauge-forming portion can be defined by the central mold parts and can have a first transverse dimension, and the shoulder-forming portions can each be defined by a respective one of the chucks and have a second transverse dimension that is larger than the first transverse dimension. A sample can be disposed in the mold cavity and solidified such that it includes a smaller-transverse-dimension gauge portion disposed between two larger-transverse-dimension shoulder portions that are disposed in the chucks.

To test the sample, the central mold parts can be decoupled from the chucks while the solidified sample's gauge portion is disposed between the chucks and its shoulder portions remain in the chucks. The chucks can be moved linearly apart to exert a tensile force on the sample, such as by coupling one of the chucks to a linear actuator and the other of the chucks to a fixed mount. Because the sample's shoulder portions are formed in the shoulder-forming portions defined by the chuck, the shoulder portions can be held in the chucks via adhesive and, optionally, vacuum forces. Each of the chucks can also include a plurality of conduits that, when the central mold parts are coupled to the chucks, are in fluid communication with the shoulder-forming portion of the mold cavity defined by the chuck and can each extend in a direction that is angularly disposed relative to (e.g., is substantially perpendicular to) a longitudinal axis that extends through the gauge-forming portion and the shoulder-forming portions of the mold cavity. Accordingly, when the sample is disposed in the mold cavity, the sample can flow into the conduits of each of the chucks to, when solidified, form branches that help keep the sample's shoulder portions in the chucks during the tensile test. The chucks' ability to hold the shoulder portions, by obviating the need for clamps that would otherwise induce deformation and stress concentrations in the shoulder portions, mitigates the risk of unintentional failure occurring in the shoulder portions such that deformation and failure can instead occur in the smaller-transverse-dimension gauge portion. This, in turn, can promote reliability and consistency in the tensile test.

Any suitable sample can be formed by the mold and tested. The use of a mold with sample-holding chucks can be particularly well suited for tensile testing of a soft tissue sample, such as solidified blood. For example, the blood can be solidified in the mold, where fibrinogen in the blood is converted to fibrin that forms of network of fibers that trap blood cells and platelets to form a solid clot. The mechanical properties of the clot can thus be reliably and consistently ascertained via a tensile test using the chucks to hold the clot, which can provide insight into how the clot may respond to instruments like a stent-retriever.

Some of the present molds include two chucks and two or more central mold parts, and some of the present systems comprise a mold having two chucks and two or more central mold parts. In some embodiments, the central mold parts are configured to be removably coupled to the chucks such that the central mold parts extend between the chucks. In some embodiments, when the central mold parts are coupled to the chucks, the central mold parts and the chucks define a mold cavity. Some of the present methods of testing a sample comprise disposing the sample in a mold cavity defined by two chucks and two or more central mold parts that are removably coupled to and extend between the chucks.

### BRIEF DESCRIPTION OF DRAWINGS

The following drawings illustrate by way of example and not limitation. For the sake of brevity and clarity, every feature of a given structure is not always labeled in every figure in which that structure appears. Identical reference numbers do not necessarily indicate an identical structure. Rather, the same reference number may be used to indicate a similar feature or a feature with similar functionality, as may non-identical reference numbers.

FIG. 1A is a perspective view of one of the present molds that includes two chucks and four central mold parts that are configured to be removably coupled to and extend between the chucks.

FIG. 1B is top view of the mold of FIG. 1A.

FIG. 1C is bottom view of the mold of FIG. 1A.

FIG. 1D is right view of the mold of FIG. 1A.

FIG. 1E is left view of the mold of FIG. 1A.

FIG. 1F is front view of the mold of FIG. 1A.

FIG. 1G is rear view of the mold of FIG. 1A.

FIG. 1H is sectional view of the mold of FIG. 1A taken along lines 1H-1H of FIG. 1B, 1I-1I of FIG. 1D, and 1J-1J of FIG. 1B, and illustrates the geometry of a mold cavity defined by the chucks and central mold parts when the central mold parts are coupled to the chucks.

FIG. 1I is sectional view of the mold of FIG. 1A taken along lines 1H-1H of FIG. 1B, 1I-1I of FIG. 1D, and 1J-1J of FIG. 1B, and illustrates the geometry of a mold cavity defined by the chucks and central mold parts when the central mold parts are coupled to the chucks.

FIG. 1J is sectional view of the mold of FIG. 1A taken along lines 1H-1H of FIG. 1B, 1I-1I of FIG. 1D, and 1J-1J of FIG. 1B, and illustrates the geometry of a mold cavity defined by the chucks and central mold parts when the central mold parts are coupled to the chucks.

FIG. 2A is perspective view of one of the chucks of the mold of FIG. 1A.

FIG. 2B is front view of one of the chucks of the mold of FIG. 1A.

FIG. 2C is side view of one of the chucks of the mold of FIG. 1A.

FIG. 2D is top view of one of the chucks of the mold of FIG. 1A.

FIG. 3A is perspective view of the central mold parts of the mold of FIG. 1A.

FIG. 3B is front view of the central mold parts of the mold of FIG. 1A.

FIG. 3C is perspective view of one of the central mold parts of the mold of FIG. 1A.

FIG. 3D is front view of one of the central mold parts of the mold of FIG. 1A.

FIG. 3E is top view of one of the central mold parts of the mold of FIG. 1A.

FIG. 4A is perspective view of an assembly including two of the central mold parts of the mold of FIG. 1A coupled to and extending between the chucks of the mold.

FIG. 4B is top view of an assembly including two of the central mold parts of the mold of FIG. 1A coupled to and extending between the chucks of the mold.

FIG. 4C is perspective view of the assembly of FIGS. 4A and 4B after the remaining two central mold parts of the mold of FIG. 1A are coupled to and extend between the chucks.

FIG. 4D is top view of the assembly of FIGS. 4A and 4B after the remaining two central mold parts of the mold of FIG. 1A are coupled to and extend between the chucks.

FIG. 5A illustrates a process for disposing and solidifying a sample in the mold cavity of the mold of FIG. 1A that comprises placing the assembly of FIGS. 4A and 4B in a container.

FIG. 5B illustrates a process for disposing and solidifying a sample in the mold cavity of the mold of FIG. 1A that comprises dispensing the sample in the container.

FIG. 5C illustrates a process for disposing and solidifying a sample in the mold cavity of the mold of FIG. 1A that comprises coupling the remaining central mold parts of the mold to the chucks.

FIG. 5D illustrates a process for disposing and solidifying a sample in the mold cavity of the mold of FIG. 1A that comprises removing the assembled mold from the container.

FIG. 6A is side view of a solidified sample formed in the mold of FIG. 1A.

FIG. 6B is top view of a solidified sample formed in the mold of FIG. 1A.

FIG. 6C is front view of a solidified sample formed in the mold of FIG. 1A.

FIG. 7A is a top view of one of the present systems that includes the mold of FIG. 1A, a linear actuator configured to be coupled to a first one of the chucks of the mold, and a mount configured to be coupled to a second one of the chucks.

FIG. 7B illustrates a process for tensile testing of a sample solidified in the mold of FIG. 1A using the system of FIG. 7A, the process including removing the central mold parts from the chucks.

FIG. 7C illustrates a process for tensile testing of a sample solidified in the mold of FIG. 1A using the system of FIG. 7A, the process including linearly moving the first chuck relative to the second chuck using the actuator.

FIG. 7D illustrates a process for tensile testing of a sample solidified in the mold of FIG. 1A using the system of FIG. 7A, the process including linearly moving the first chuck relative to the second chuck using the actuator until failure of the solidified sample.

### DESCRIPTION OF EMBODIMENTS

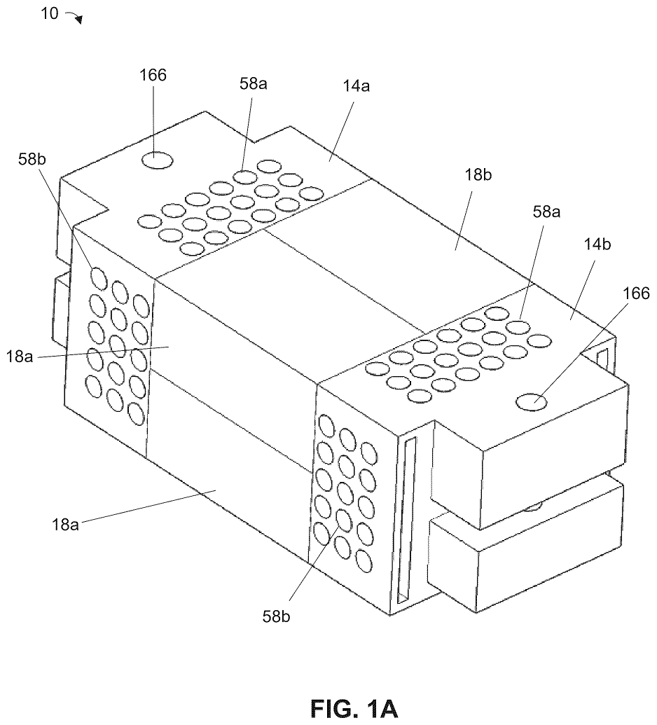

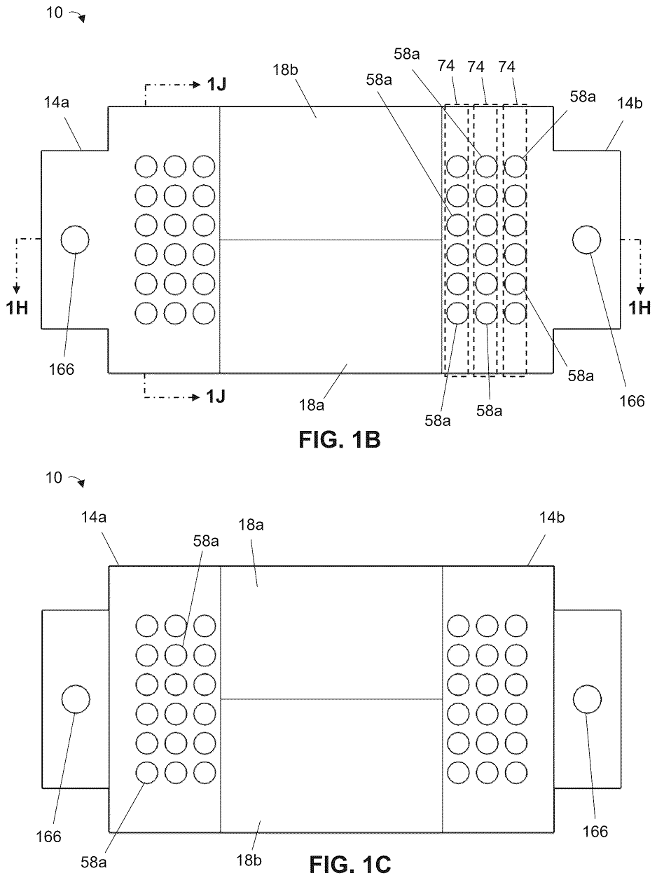

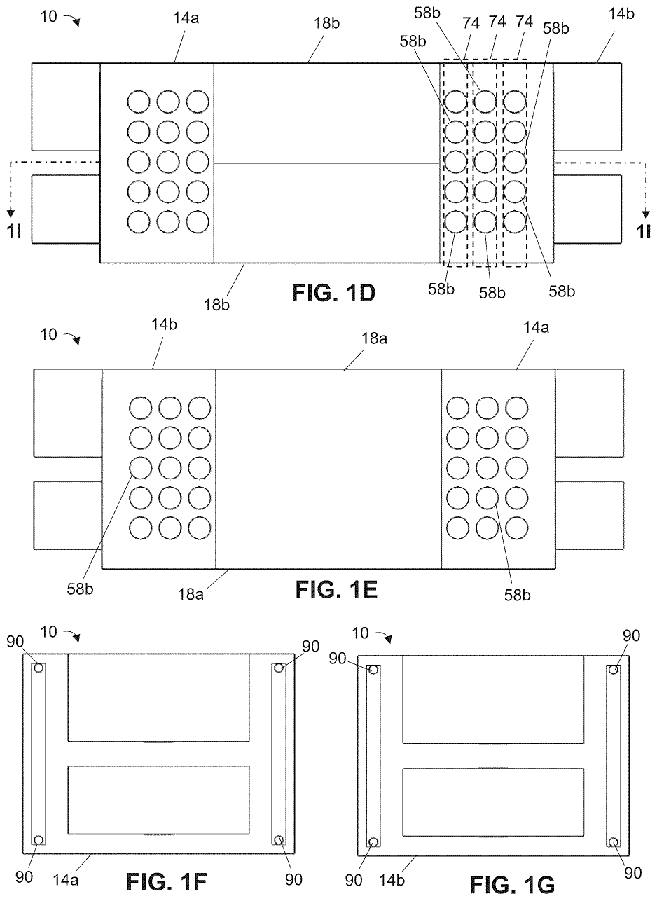

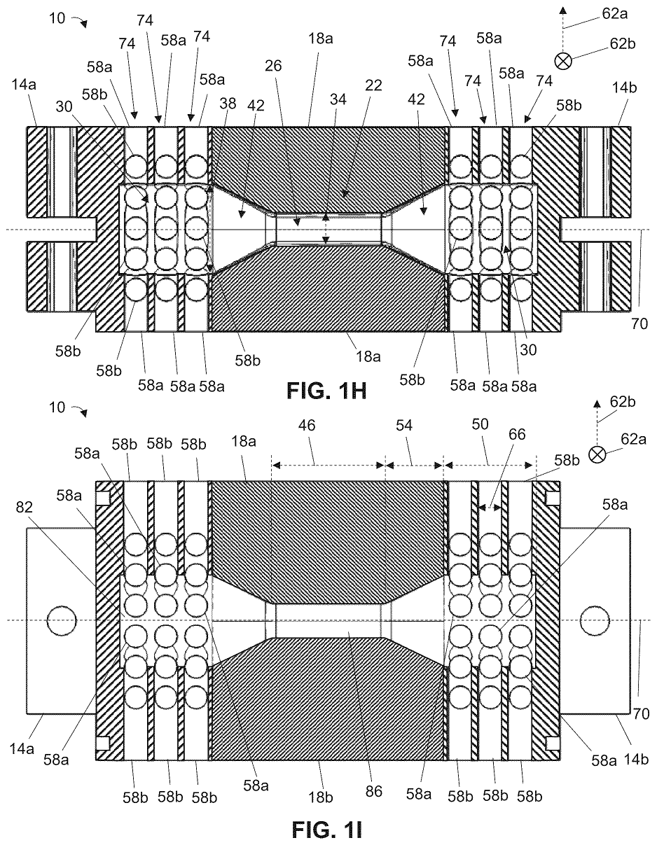

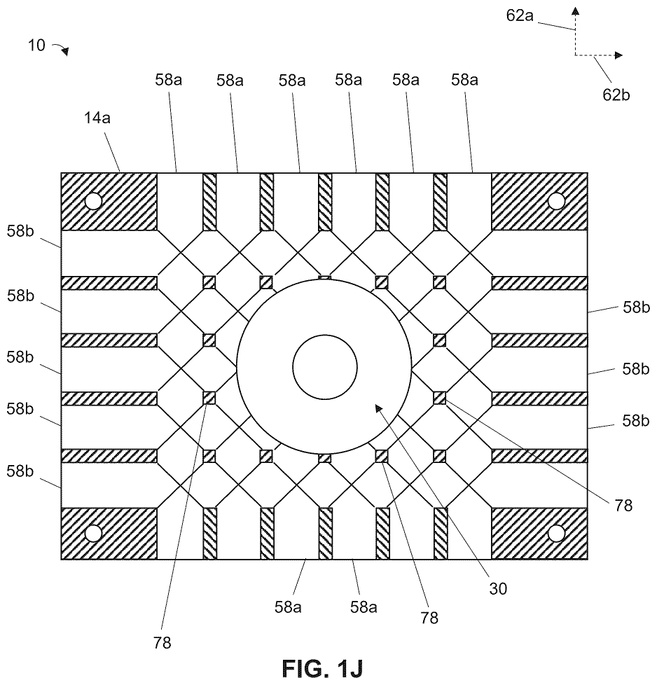

Referring to FIGS. 1A-1J, shown is an embodiment 10 of the present molds that includes two chucks 14a and 14b and two or more central mold parts 18a and 18b, such as greater than or equal to any one of, or between any two of, two, three, four, five, six, seven, or eight central mold parts; as shown, the mold comprises four central mold parts. Central mold parts 18a and 18b can be configured to be removably coupled to chucks 14a and 14b such that the central mold parts extend between the chucks. When central mold parts 18a and 18b are coupled to chucks 14a and 14b, the central mold parts and the chucks can define a mold cavity 22 (FIGS. 1H-1J) in which a sample can be solidified and shaped. As described in further detail below, after a sample is solidified and shaped in mold cavity 22, central mold parts 18a and 18b can be removed from chucks 14a and 14b and the chucks can hold the solidified sample as the chucks are pulled apart for tensile testing of the sample.

Referring specifically to FIGS. 1H-1J, mold cavity 22 can have a geometry that facilitates consistent and predictable tensile testing of a sample shaped in the mold cavity. As shown, mold cavity 22 has a gauge-forming portion 26 and two shoulder-forming portions 30, wherein the gauge-forming portion is defined by the central mold parts and each of the shoulder-forming portions is defined by a respective one of the chucks such that the gauge-forming portion is disposed between the shoulder-forming portions. Gauge-forming portion 26 can have a first transverse dimension 34 (e.g., diameter) and each of shoulder-forming portions 30 can have a second transverse dimension 38 (e.g., diameter) that is larger than the first transverse dimension, such as a second transverse dimension that is greater than or equal to any one of, or between any two of, 1.5, 1.6, 1.7, 1.8, 1.9, 2.0, 2.1, 2.2, 2.3, 2.4, 2.5, 2.6, or 2.7 times the first transverse dimension (e.g., at least 1.5 times or at least 2.0 times the first transverse dimension). As described in further detail below, with shoulder-forming portions 30 each having a second transverse dimension 38 that is larger than (e.g., at least 1.5 times or at least 2.0 times) first transverse dimension 34 of gauge-forming portion 26, shoulder portions of a sample shaped in mold cavity 22's shoulder-forming portions can each have a larger transverse dimension than a gauge portion of the sample shaped in the mold cavity's gauge-forming portion. During tensile testing in which the sample is held in chucks 14a and 14b, this can encourage deformation and failure of the sample in the smaller-transverse-dimension gauge portion while promoting adhesion of the sample's larger-transverse-dimension shoulder portion to the chucks (e.g., because the larger second transverse dimension can yield a larger shoulder surface area in contact with the chucks), which can promote consistency and reliability in the tensile test (e.g., by mitigating the risk of deformation and failure in the shoulder portions). The transverse dimension of each of gauge-forming portion 26 and shoulder-forming portions 30 can be substantially constant along a length 46 or 50 of the portion (e.g., each of the portions can be cylindrical).

As shown, for each of chucks 14a and 14b, shoulder-forming portion 30 does not extend to an exterior of mold 10 when central mold parts 18a and 18b are coupled to the chucks. Air thus cannot enter shoulder-forming portions 30 defined by chucks 14a and 14b once a sample disposed therein is solidified, which creates a vacuum within the shoulder-forming portions. Accordingly, when central molds parts 18a and 18b are removed from chucks 14a and 14b to expose the non-shoulder portions of the solidified sample to the ambient environment, for each of the chucks, the pressure differential between the ambient environment and the vacuum in the shoulder-forming portion urges the shoulder portion of the sample into the chuck to help maintain the shoulder portion in the chuck during a tensile test. In other embodiments, however, shoulder-forming portion 30 of each of chucks 14a and 14b can extend to the exterior of mold 10.

Mold cavity 22 can further comprise two expanding portions 42 that are each defined by central mold parts 18a and 18b and extend between gauge-forming portion 26 and a respective one of shoulder-forming portions 30. Expanding portions 42 of mold cavity 22 can shape expanding portions of a sample disposed therein, which can function as transitions between the sample's smaller-transverse-dimension gauge portion and larger-transverse-dimension shoulder portions to, during a tensile test, further promote deformation and failure within the gauge portion and mitigate the risk of the same occurring in the shoulder portions. To do so, each of expanding portions 42 can have a transverse dimension (e.g., diameter) that increases moving from gauge-forming portion 26 (e.g., where the expanding portion's transverse dimension can be first transverse dimension 34) to the respective shoulder-forming portion 30 (e.g., where the expanding portion's transverse dimension can be second transverse dimension 38). As shown, each of expanding portions 42 is frustoconical to define an expanding transition between gauge-forming portion 26 and a respective one of shoulder-forming portions 30.

Gauge-forming portion 26, shoulder-forming portions 30, and expanding portions 42 of mold cavity 22 can have any suitable dimensions for shaping and testing a sample. Preferably, those portions of mold cavity 22 are relatively compact such that mold 10 is of a manageable size for testing and to reduce the amount of sample needed to fill the mold cavity, which can be particularly beneficial for testing samples like blood where it can be desirable (e.g., for a subject's comfort) to collect smaller volumes of the sample. To illustrate, first transverse dimension 34 of gauge-forming portion 26 can be less than or equal to any one of, or between any two of, 12, 11, 10, 9, 8, 7, or 6 millimeters (mm) (e.g., between 5 and 10 mm) and second transverse dimension 48 of each of shoulder-forming portions 30 can be less than or equal to any one of, or between any two of, 40, 35, 30, 25, 20, or 15 mm (e.g., between 15 and 25 mm). Furthermore, length 46 of gauge-forming portion 26 (e.g., measured in a direction aligned with a longitudinal axis 70 that extends through the gauge-forming portion and shoulder forming portions 30) can be less than or equal to any one of, or between any two of, 45, 40, 35, 30, 25, 20, or 15 mm (e.g., between 15 and 35 mm). Length 50 of each of shoulder-forming portions 30 (e.g., measured in a direction aligned with longitudinal axis 70) can likewise be compact but also of a length that yields an adequate surface area for a sample's shoulder portion shaped in mold cavity 22's shoulder-forming portion to promote adhesion with the chuck (e.g., 14a or 14b) defining the shoulder-forming portion. For example, length 50 can be less than or equal to any one of, or between any two of, 40, 35, 30, 25, 20, or 15 mm (e.g., between 15 and 30 mm). And length 54 of each of expanding portions can be smaller than lengths 46 and 50 of gauge-forming portion 26 and shoulder-forming portions 30, respectively, such as less than or equal to any one of, or between any two of, 25, 22.5, 20, 17.5, 15, 12.5, 10, or 7.5 mm (e.g., between 7.5 and 20 mm).

To further promote the ability of chucks 14a and 14b to hold a sample during a tensile test such that sample failure occurs at the gauge portion rather than at one of the shoulder portions, each of the chucks can comprise a plurality of conduits 58a and 58b. For each of chucks 14a and 14b, conduits 58a and 58b can be in fluid communication with shoulder-forming portion 30 defined by the chuck such that a sample disposed in mold cavity 22 can also flow into the conduits and, when solidified, include branches in the conduits that are coupled to the sample's shoulder portion. Each of conduits 58a and 58b can extend in a direction that is angularly disposed relative to longitudinal axis 70, such as in a direction that is substantially perpendicular to the longitudinal axis. Accordingly, when chucks 14a and 14b are pulled apart along longitudinal axis 70 during a tensile test, the sample branches formed in the angular-disposed conduits 58a and 58b can resist forces tending to pull the sample out of the chucks to help maintain the sample in the chucks.

Preferably, conduits 58a and 58b of each of chucks 14a and 14b include a plurality of first conduits 58a that each extend in a first direction 62a and a plurality of second conduits 58b that each extend in a second direction 62b that is substantially perpendicular to the first direction. For example, each of chucks 14a and 14b can comprise greater than or equal to any one of, or between any two of, five, six, seven, eight, nine, ten, eleven, twelve, thirteen, fourteen, fifteen, sixteen, seventeen, eighteen, nineteen, or twenty (e.g., at least ten) first conduits 58a and greater than or equal to any one of, or between any two of, five, six, seven, eight, nine, ten, eleven, twelve, thirteen, fourteen, fifteen, sixteen, seventeen, eighteen, nineteen, or twenty (e.g., at least ten) second conduits 58b. Including conduits 58a and 58b extending in both first and second directions 62a and 62b can allow the formation of more sample branches to help keep the sample's shoulder portions in chucks 14a and 14b during tensile testing. Furthermore, for each of chucks 14a and 14b, first and second conduits 58a and 58b can be arranged such that the conduits include one or more sets 74 of conduits, such as greater than or equal to any one of, or between any two of, one, two, three, four, five, six, or seven sets of conduits, that each comprise two or more of the first conduits and two or more of the second conduits (FIGS. 1B and 1D); as shown, each of the chucks includes three sets of conduits. For each of set(s) 74 of conduits 58a and 58b, the first and second conduits of the set can be coplanar and each of the first conduits of the set can intersect at least one of the second conduits of the set. With such an arrangement, the body of each of chucks 14a and 14b can include a plurality of portions 78 that are each circumscribed by two of first conduits 58a and two of second conduits 58b (FIG. 1J) such that sample branches formed in those circumscribing conduits can define a loop that interfaces with the chuck to promote the chuck's ability to robustly and stably hold the shoulder portion of the sample.

Conduits 58a and 58b can each have a transverse dimension 66 (e.g., diameter) that is relatively small to allow more conduits to be defined by chucks 14a and 14b and thus more sample branches to be formed in the chucks, which can promote the chucks' ability to hold the shoulder portions of the sample. However, sample flow-such as the flow of blood-through conduits 58a and 58b for formation of the sample branches can be impeded (e.g., by trapped bubbles) when the conduits each have a small transverse dimension 66. To balance these considerations, transverse dimension 66 of each of conduits 58a and 58b can be less than or equal to any one of, or between any two of 50%, 45%, 40%, 35%, 30%, 25%, or 20%—while still being at least 10% or at least 15%—of shoulder-forming portion 30's transverse dimension 58a and/or length 50 (e.g., between 15% and 35% of the shoulder-forming portion's transverse dimension and/or length). For example, transverse dimension 66 of each of conduits 58a and 58b can be less than or equal to any one of, or between any two of, 12, 11, 10, 9, 8, 7, 6, 5, 4, or 3 mm (e.g., between 3 and 8 mm).

As shown, for each of chucks 14a and 14b, each of conduits 58a and 58b can extend to an exterior of the chuck. This can facilitate the disposal of a sample in mold cavity 22 and conduits 58a and 58b, especially when shoulder-forming portions 30 do not extend to an exterior of mold 10, by allowing, for example, the evacuation of air as the sample is disposed in the mold cavity.

Because chucks 14a and 14b serve to hold the solidified sample during a tensile test, each of the chucks can have one or more interior surfaces 82—wherein shoulder-forming portion 30 is defined by at least one of the chuck's interior surface(s) and each of conduits 58a and 58b is defined by at least one of the chuck's interior surface(s)—that can promote the chuck's ability to hold the sample. Furthermore, because central mold parts 18a and 18b are removed from chucks 14a and 14b for tensile testing, each of the central mold parts can have one or more interior surfaces 86—wherein the central mold parts' interior surfaces cooperate to define gauge-forming portion 26 and expanding portions 42 of mold cavity 22—that can promote the central mold part's ability to be released from the sample. For example, interior surface(s) 82 of each of chucks 14a and 14b can have a higher surface roughness than interior surface(s) 86 of each of central mold parts 18a and 18b. As an illustration, an average maximum profile peak height of each of interior surface(s) 82 of chucks 14a and 14b (e.g., measured along length 50 of shoulder-forming portion 30, for the interior surface(s) defining the shoulder-forming portion, or along a length of a conduit 58a or 58b, for the interior surface(s) defining that conduit) can be greater than or equal to any one of, or between any two of, 0.06, 0.07, 0.08, 0.09, 0.10, 0.11, 0.12, 0.13, or 0.14 mm. Furthermore, interior surface(s) 86 of each of central mold parts 86 can comprise a hydrophobic material such as acrylic that can facilitate the mold parts' release from the sample and promote the smoothness of the interior surface(s) (e.g., when applied as a surface coating).

Mold 10's chucks 14a and 14b and central molds parts 18a and 18b can be made of any suitable material. Because the sample shaped by mold 10 can be a biological sample such as blood, the mold can be a disposable product that preferably is made of cost-effective materials. For example, chucks 14a and 14b and central mold parts 18a and 18b can each comprise a polymeric material such as acrylonitrile butadiene styrene (ABS), polylactic acid (PLA), polyethylene terephthalate glycol (PETG), polyethylene terephthalate (PET), high-impact polystyrene (HIPS), thermoplastic polyurethane (TPU), and/or aliphatic polyamides (nylon). Chucks 14a and 14b and central mold parts 18a and 18b can each be 3D printed (e.g., using fused deposition modeling), which can facilitate cost-effective production and promote the surface roughness of the chucks' interior surfaces 82 for holding the sample.

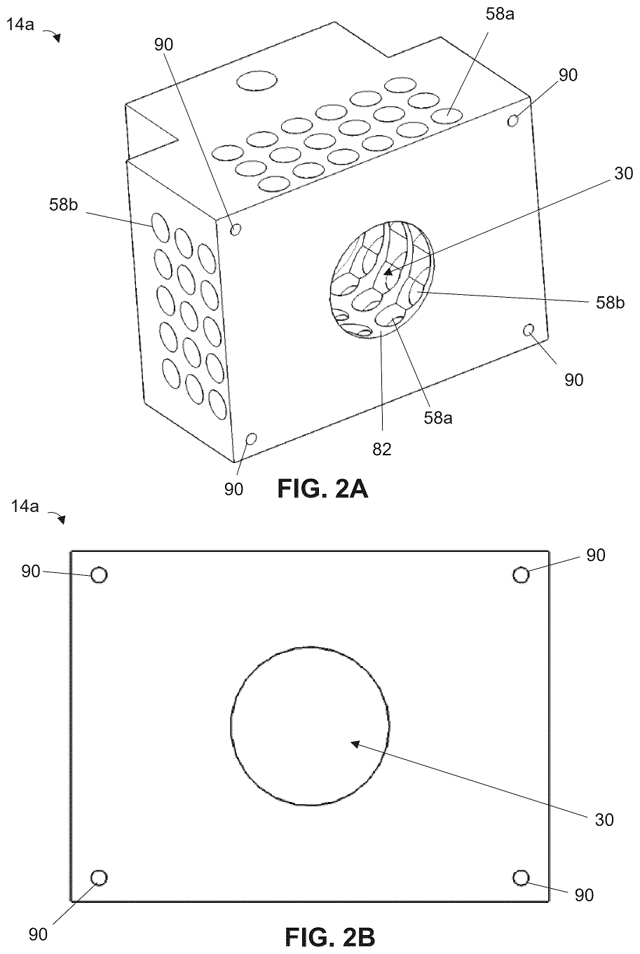

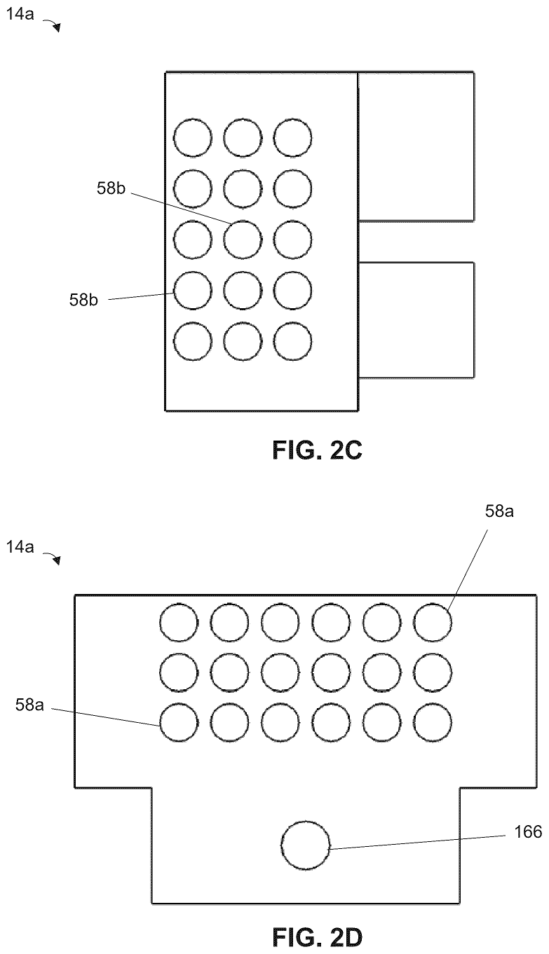

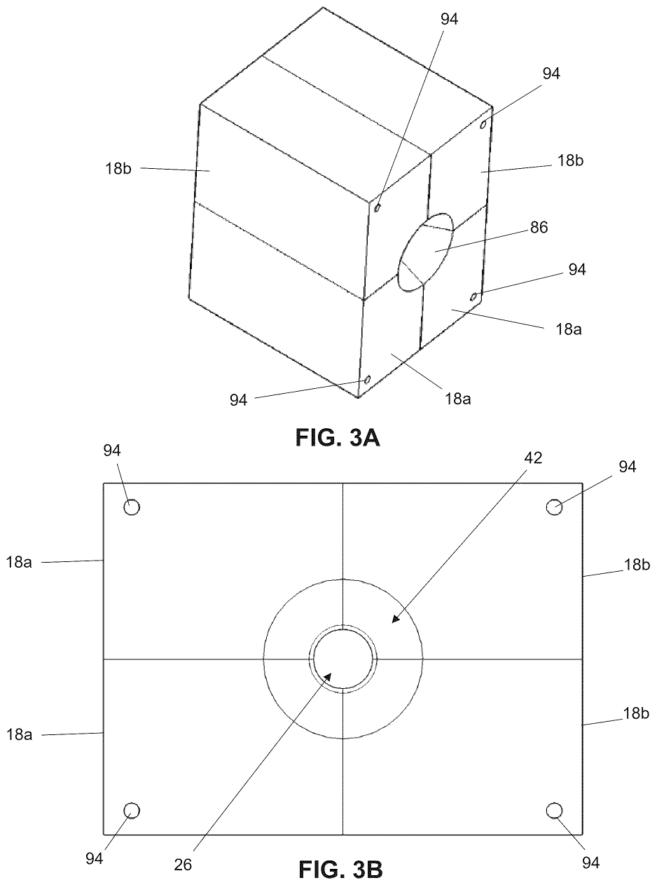

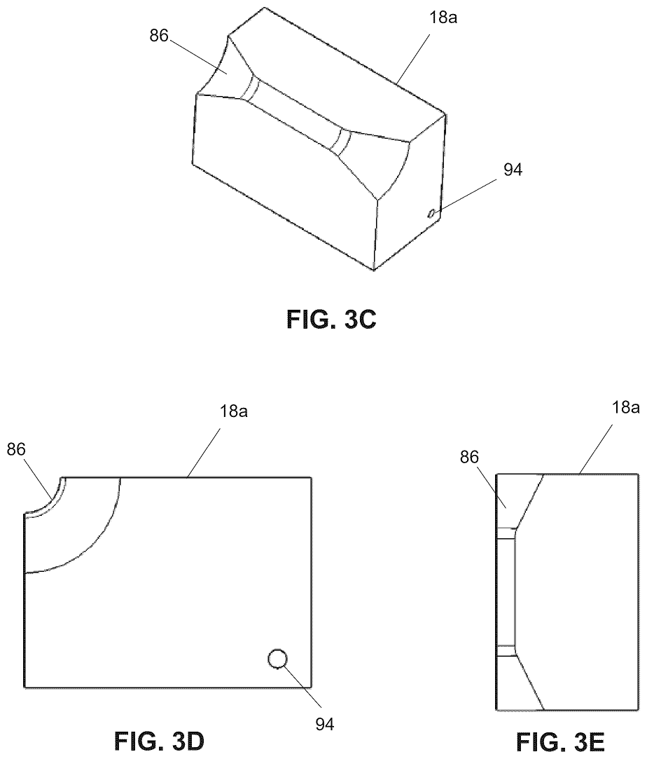

As explained above, central mold parts 18a and 18b can be configured to be removably coupled to chucks 14a and 14b. Such removable coupling can be achieved in any suitable manner. For example, and referring additionally to FIGS. 2A-2D and FIGS. 3A-3E, each of chucks 14a and 14b can include two or more openings 90 extending therethrough (e.g., in a direction substantially aligned with longitudinal axis 70)—such as at least one opening for each of central mold parts 18a and 18b—and each of the central mold parts can include one or more openings 94 extending therethrough (e.g., in a direction substantially aligned with longitudinal axis 70). Opening(s) 94 of each of central mold parts 18a and 18b, when the central mold part extends between first and second chucks 14a and 14b, can be aligned with a respective one of openings 90 of the first chuck and a respective one of the openings of the second chuck such that a coupling member such as wire or a fastener like a pin can be inserted through the openings of the central mold part and the chucks to couple them together. Each of central mold parts 18a and 18b can be removed from chucks 14a and 14b at least by removing the coupling member from aligned openings 90 and 94.

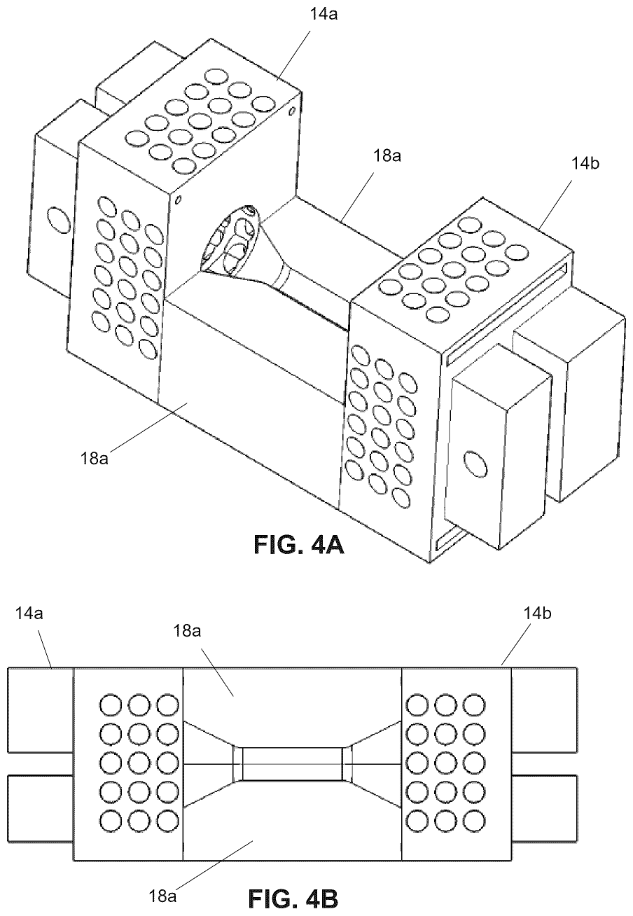

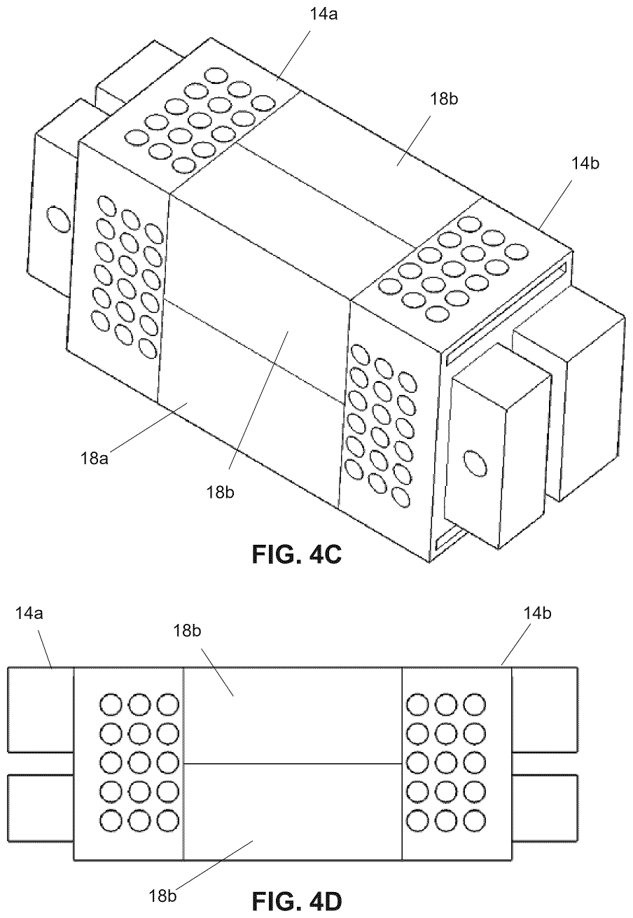

Referring to FIGS. 4A-4D, shown is a manner in which mold 10 can be assembled. As shown, not all of central mold parts 18a and 18b need to be coupled to chucks 14a and 14b at the same time. For example, central mold parts 18a and 18b can include one or more first central mold parts 18a and one or more second central mold parts 18b, such as greater than or equal to any one of, or between any two of, one, two, three, or four first central mold parts and greater than or equal to any one of, or between any two of, one, two, three, or four second central mold parts; as shown, mold 10 includes two first central mold parts and two second central mold parts. First central mold part(s) 18a can be coupled to chucks 14a and 14b (e.g., in the manner described above, such as with a wire extending through openings 90 and 94 of the chucks and first central mold parts) before second central mold part(s) 18b are coupled to the chucks (FIGS. 4A and 4B). As explained in further detail below, a sample can be dispensed onto this assembly before the coupling of second central mold part(s) 18b to chucks 14a and 14b (FIGS. 4C and 4D), which can facilitate disposal of the sample in mold cavity 22.

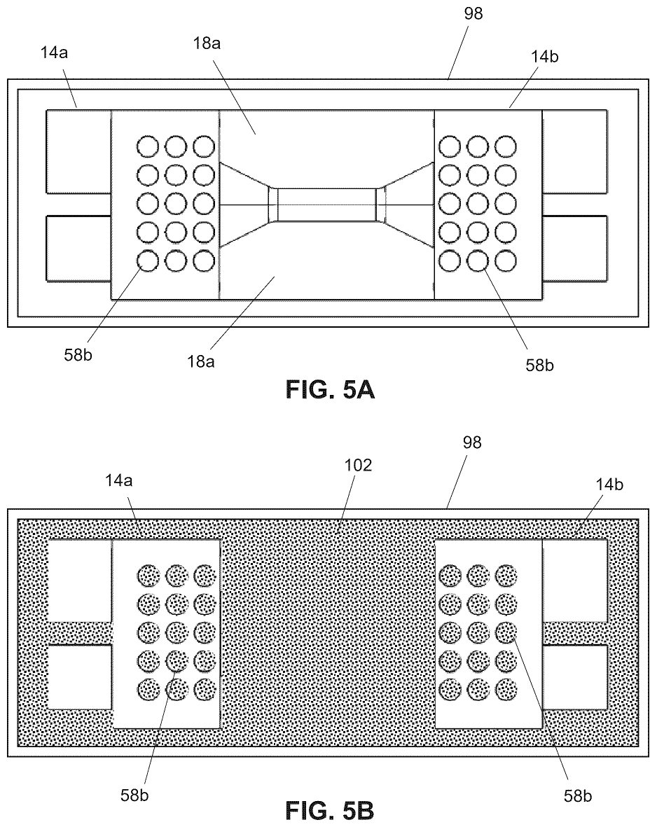

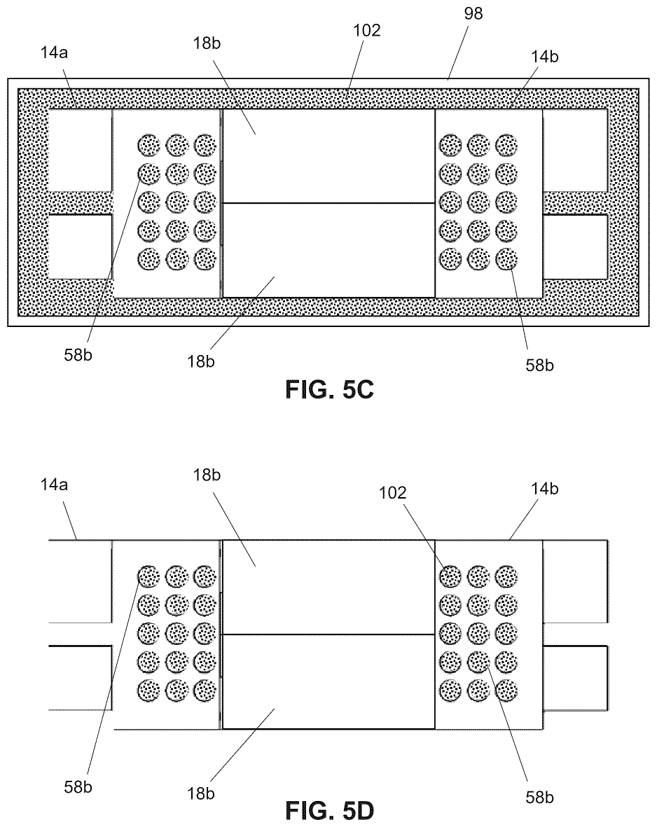

Referring to FIGS. 5A-5C, some of the present methods of testing a sample (e.g., 102) comprise disposing the sample in a mold cavity (e.g., 22) defined by two chucks (e.g., 14a and 14b) and two or more central mold parts (e.g., 18a and 18b) that are removably coupled to and extend between the chucks (e.g., any of the above-described mold cavities defined by any of the above-described chucks and central mold parts), optionally such that the sample flows into conduits (e.g., 58a and 58b) of each of the chucks. The sample can be a material whose mechanical material properties are under investigation, and preferably is a material that is compressible when solidified; as described in further detail below, the systems and methods described herein are particularly well-suited for tensile testing of such compressible-when-solidified materials. As used herein, “compressible materials” refers to materials that, when solidified, are prone to significant deformation and induced stress concentrations when held by conventional clamping grips. For example, the systems and methods described herein may be especially advantageous when testing soft tissue like blood, which can be solidified to form a clot for testing.

The sample can be disposed in the mold cavity in any suitable manner. In the embodiment shown, disposing the sample in the mold cavity can comprise coupling the one or more first central mold parts (e.g., 18a) to the chucks such that the first central mold part(s) extend between the chucks as explained above for FIGS. 4A and 4B. The chucks and the first central mold part(s) can then be placed in a container (e.g., 98) (FIG. 5A) and the sample can be dispensed into the container while the chucks and the first central mold part(s) are disposed in the container and the first central mold part(s) are coupled to the chucks (FIG. 5B). As shown, the sample can be disposed in the container such that the sample submerges the first central mold part(s) and enters shoulder-forming portion (e.g., 30) and at least some of the conduits defined by each of the chucks. After the sample is dispensed in the container, the one or more second central mold parts (e.g., 18b) can be coupled to the chucks such that the second central mold part(s) extend between the chucks (FIG. 5C) to define the mold cavity that accordingly contains the sample. To the extent that the sample did not completely fill the shoulder-forming portions and conduits of the chucks when only the first central mold part(s) were coupled to the chucks, the coupling of the second central mold part(s) to the chucks can displace the sample such that the sample completely fills the mold cavity and the conduits. While as shown disposing the sample in the mold cavity includes sequential coupling of the first central mold part(s) and second central mold part(s), in other embodiments all of the central mold parts can be coupled to the chucks to define the mold cavity and the sample can be introduced into the mold cavity after all of the central mold parts are coupled together.

Some methods comprise a step of solidifying the sample while the sample is disposed in the mold cavity (and optionally while the sample is disposed in the container). Solidification can be performed in any suitable manner. For example, when the sample comprises blood, solidifying the sample can comprise adding one or more clotting agents to the sample. A blood sample may include one or more anticoagulants like citric acid, and the clotting agent(s) can include one or more compounds such as calcium chloride that reverse the effect of those anticoagulants. The clotting agent(s) can also include thrombin, which is an enzyme that can facilitate the conversion of fibrinogen in the blood to fibrin, which forms a network of fibers that trap blood cells and platelets to form a solid clot. If the sample is disposed in the mold cavity via sequential coupling of the first central mold part(s) and the second central mold part(s) to the chucks as described above, the clotting agent(s) are preferably added to the sample before the second central mold part(s) are coupled to the chucks to facilitate the dispersion thereof throughout the sample. Solidifying the sample can also comprise heating the sample, such as in a heated chamber having a temperature that is greater than or equal to any one of, or between any two of 34, 35, 36, 37, 38, 39, or 40° C., optionally for greater than or equal to any one of, or between any two of, 10.0, 12.5, 15.0, 17.5, 20.0, 22.5, 25.0, 27.5, 30.0, 32.5, 35.0, 37.5, or 40.0 minutes (e.g., between 15 and 30 minutes). When the sample is blood, the heating can facilitate the clotting process in which fibrinogen is converted into fibrin for solidification. After solidification, excess sample can be removed from the exterior of the mold, such as after removing the mold from the container (FIG. 5D).

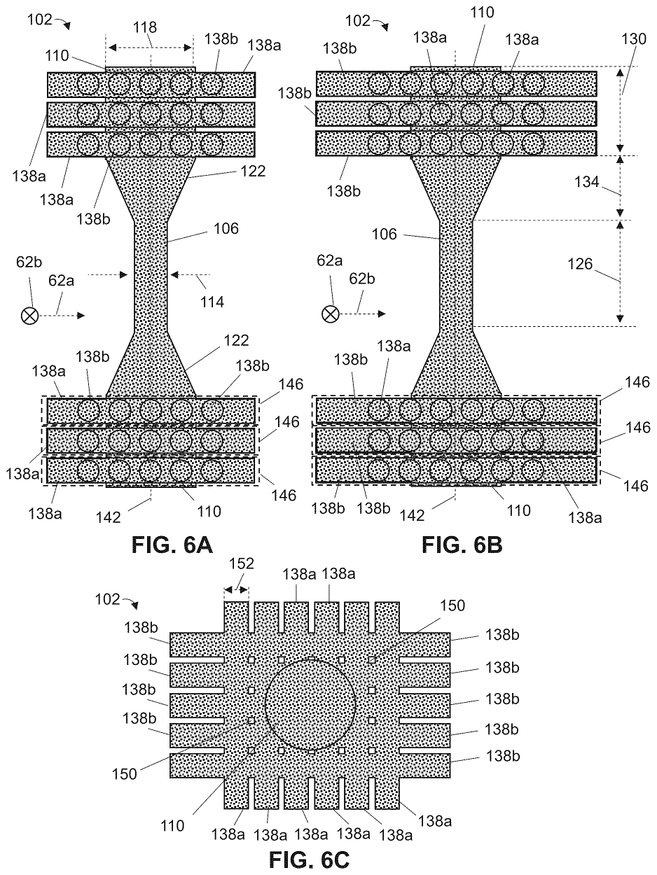

Referring to FIGS. 6A-6C, shown is a sample 102 that can be formed in the above-described process. Sample 102 can have a body that can be compressible (e.g., comprising solidified blood that can include fibrin) and that can include a gauge portion 106 that can be formed in mold cavity 22's gauge-forming portion 26 and two shoulder portions 110 that are each formed in a respective one of the mold cavity's shoulder-forming portions 30, wherein the gauge portion is disposed between the shoulder portions. The body of sample 102 can also include two expanding portions 122 that each extend between gauge portion 106 and a respective one of shoulder portions 110. Because sample 102's gauge portion 106, shoulder portions 110, and expanding portions 122 are formed by mold cavity 22's gauge-forming portion 26, shoulder-forming portions 30, and expanding portions 42, respectively, the geometry and dimensions of those portions of the sample can be the same as the geometry and dimensions of those portions of the mold cavity. For example, gauge portion 106 can be cylindrical and can have a first transverse dimension 114 (e.g., diameter)—which can be, for example, less than or equal to any one of, or between any two of, 12, 11, 10, 9, 8, 7, or 6 millimeters (mm) (e.g., between 5 and 10 mm)—and each of shoulder portions 110 can be cylindrical and can have a second transverse dimension 118 (e.g., diameter)—which can be, for example, less than or equal to any one of, or between any two of, 40, 35, 30, 25, 20, or 15 mm (e.g., between 15 and 25 mm)—that is larger than the first transverse dimension, such as a second transverse dimension that is greater than or equal to any one of, or between any two of, 1.5, 1.6, 1.7, 1.8, 1.9, 2.0, 2.1, 2.2, 2.3, 2.4, 2.5, 2.6, or 2.7 times the first transverse dimension (e.g., at least 1.5 times or at least 2.0 times the first transverse dimension). Expanding portions 122 can each be frustoconical and can each have a transverse dimension (e.g., diameter) that increases moving from gauge portion 106 (e.g., where the expanding portion's transverse dimension can be first transverse dimension 114) to the respective shoulder portion 110 (e.g., where the expanding portion's transverse dimension can be second transverse dimension 118). Likewise, length 126 of gauge portion 106 (e.g., measured in a direction aligned with a longitudinal axis 142 that extends through the gauge portion and shoulder portions 110) can be less than or equal to any one of, or between any two of, 45, 40, 35, 30, 25, 20, or 15 mm (e.g., between 15 and 35 mm), length 130 of each of shoulder portions 110 (e.g., measured in a direction aligned with longitudinal axis 142) can be less than or equal to any one of, or between any two of, 40, 35, 30, 25, 20, or 15 mm (e.g., between 15 and 30 mm), and length 134 of each of expanding portions 122 can be smaller than the lengths of the gauge portion and shoulder portions, such as less than or equal to any one of, or between any two of, 25, 22.5, 20, 17.5, 15, 12.5, 10, or 7.5 mm (e.g., between 7.5 and 20 mm). As explained above, such sizing can promote adhesive forces between shoulder portions 110 and chucks 14a and 14b that hold the shoulder portions, encourage failure in the smaller-transverse-dimension gauge portion 106 of sample 102 during a tensile test for consistent, reliable test results, and allow formation of a manageably-sized solidified sample with a relatively low volume of material.

Additionally, when each of chucks 14a and 14b includes conduits 58a and 58b, sample 102 can comprise, for each of shoulder portions 110, a plurality of branches 138a and 138b that can be formed in the conduits and thus can be coupled to the shoulder portion and extend in a direction that is angularly disposed relative to (e.g., substantially perpendicular to) longitudinal axis 142. As explained above, branches 138a and 138b can help maintain sample 102's shoulder portions 110 in chucks 14a and 14b during a tensile test to mitigate the risk of unintended deformation or failure in the shoulder portions. Because branches 138a and 138b are formed in conduits 58a and 58b, they can have the same arrangement and dimensions as the conduits. For example, branches 138a and 138b can include a plurality of first branches 138a—such as greater than or equal to any one of, or between any two of, five, six, seven, eight, nine, ten, eleven, twelve, thirteen, fourteen, fifteen, sixteen, seventeen, eighteen, nineteen, or twenty (e.g., at least ten) first branches—that each extend in first direction 62a and a plurality of second branches 138b—such as greater than or equal to any one of, or between any two of, five, six, seven, eight, nine, ten, eleven, twelve, thirteen, fourteen, fifteen, sixteen, seventeen, eighteen, nineteen, or twenty (e.g., at least ten) second branches—that each extend in a second direction 62b that is substantially perpendicular to the first direction, optionally such that there are one or more sets 146, such as greater than or equal to any one of, or between any two of, one, two, three, four, five, six, or seven sets (e.g., three sets), of branches that each comprise two or more of the first branches and two or more of the second branches. As with set(s) 74 of conduits 58a and 58b, for each of set(s) 146 of branches 138a and 138b, the first and second branches of the set can be coplanar and each of the first branches can intersect at least one of the second branches of the set. With this intersection, each of shoulder portions 110 can include, for each of set(s) 146 of branches 138a and 138b, a plurality of openings 150 disposed in the shoulder portion that are each circumscribed by two of the first branches of the set and two of the second branches of the set. As described above, the circumscribing first and second branches 138a and 138b can thus define a loop around portion 78 of the body of the chuck 14a or 14b that the branches are disposed in to promote a stable and robust interface between sample 102's shoulder portion 110 and the chuck. Furthermore, each of branches 138a and 138b can have a transverse dimension 152 (e.g., diameter) that can be less than or equal to any one of, or between any two of 50%, 45%, 40%, 35%, 30%, 25%, or 20%—while still being at least 10% or at least 15%—of shoulder portion 110's transverse dimension 118 and/or length 130 (e.g., between 15% and 35% of the shoulder-forming portion's transverse dimension and/or length), such as a transverse dimension that is less than or equal to any one of, or between any two of, 12, 11, 10, 9, 8, 7, 6, 5, 4, or 3 mm (e.g., between 3 and 8 mm).

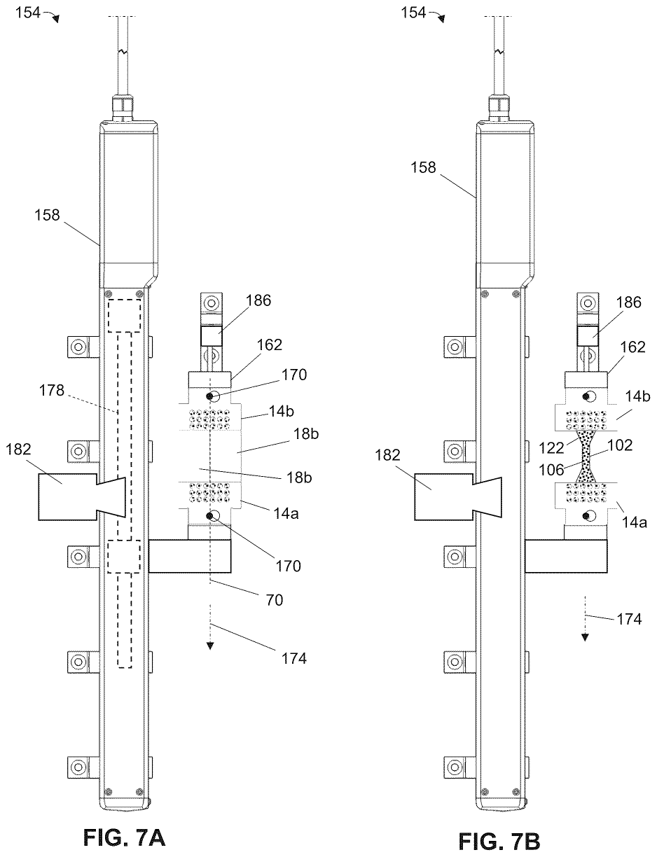

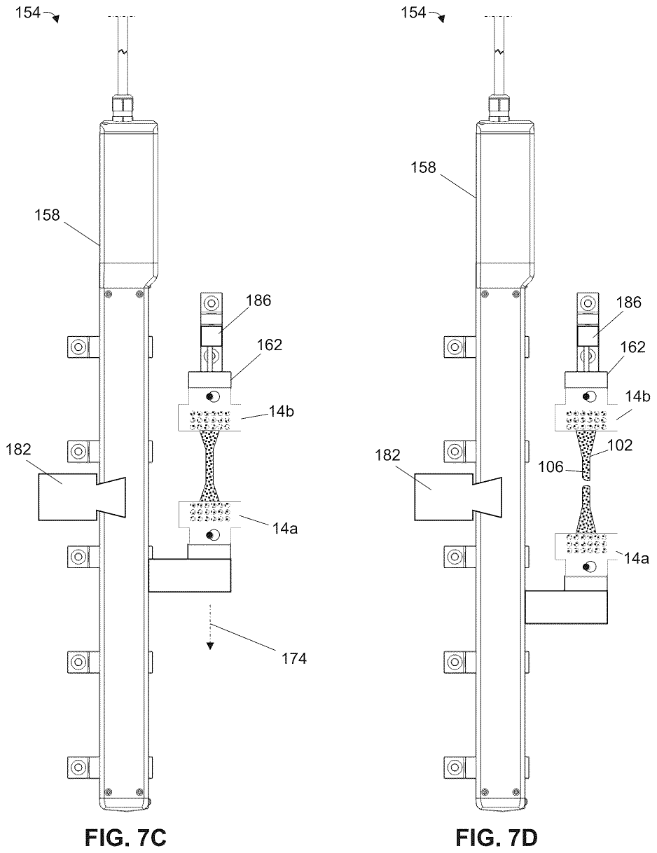

Referring to FIG. 7A, shown is a system 154 that includes mold 10 and can be used to perform tensile testing of a sample 102 shaped and solidified in the mold. As shown, system 154 can comprise an actuator 158 that can be configured to be removably coupled to a first one of mold 10's chucks 14a and a mount 162 that can be configured to be removably coupled to a second one of the mold's chucks 14b. For example, each of chucks 14a and 14b can comprise an opening 166 (FIGS. 1A-1C) through which a fastener 170 such as a pin, a bolt, and/or the like can be disposed. First chuck 14a's opening 166 can interface with an opening on actuator 158 and fastener 170 can be passed therethrough to couple the first chuck to the actuator, and second chuck 14b's opening 166 can interface with an opening on mount 162 and fastener 170 can be passed therethrough to couple the second chuck to the mount. When actuator 158 is coupled to first chuck 14a, mount 162 is coupled to second chuck 14b, and central mold parts 18a and 18b are not coupled to the chucks, the actuator can be configured to move the first chuck linearly relative to the second chuck along longitudinal axis 70 that extends through the chucks (e.g., in direction 174), which can be horizontal to mitigate the risk of portions of the sample falling and contaminating one or more components of system 154. For example, actuator 158 can be a linear actuator and can have any suitable mechanism (e.g., electromechanical, hydraulic, pneumatic, and/or the like) to cause such linear movement for a tensile test of a sample 102 held by chucks 14a and 14b.

System 154 can include one or more mechanisms for measuring the mechanical properties of a sample 102 being tested. For example, system 154 can include one or more mechanisms to measure deformation (e.g., the change in length and/or transverse dimension) of a sample 102 during a tensile test. As shown, to measure such deformation, actuator 154 can include a displacement gauge 178 that is configured to measure a distance that first chuck 14a moves relative to second chuck 14b along longitudinal axis 174 and thus the change in length in sample 102 that is held by and extends between the chucks. System 154 can also comprise one or more cameras 182 configured to capture video of a region between chucks 14a and 14b, which can in turn be used to determine a change in length of sample 102 and the change in transverse dimension of the sample's gauge portion 106. Furthermore, system 154 can include one or more mechanisms to measure a force with which sample 102 is being pulled during a tensile test. For example, system 154's mount 162 can comprise a load cell 186 that is configured to measure a force exerted on second chuck 14b—and thus on sample 102 held by chucks 14a and 14b—in a direction along longitudinal axis 174. The deformation and load data can be used to calculate a variety of mechanical properties of sample 102, such as its yield strength, ultimate tensile strength, modulus of elasticity, and elongation.

Referring additionally to FIGS. 7B-7D, some methods comprise, after the sample is solidified, decoupling the central mold parts from the chucks (FIG. 7B). With the central mold parts decoupled from the chucks, a gauge portion (e.g., 106) of the solidified sample formed in the gauge-forming portion (e.g., 26) of the mold cavity and the optional expanding portions (e.g., 122) formed in the expanding portions (e.g., 42) of the mold cavity can be disposed between the chucks. Additionally, each of the two shoulder portions (e.g., 110) of the solidified sample formed in a respective one of the shoulder-forming portions (e.g., 30) can be disposed in a respective one of the chucks such that the chucks hold the sample. As shown, the central mold parts can be decoupled from the chucks after the first chuck is coupled to an actuator (e.g., 158) and the second chuck is coupled to a mount (e.g., 162) of the system as described above (FIG. 7A).

Some methods comprise, after the central mold parts are decoupled from the chucks, linearly moving the first and second chucks apart along a longitudinal axis (e.g., 70) extending through the chucks and the solidified sample (e.g., horizontally, as described above), such as by linearly moving the first chuck relative to the second chuck along the longitudinal axis (e.g., in direction 174) using the actuator after coupling the first chuck to the actuator and the second chuck to the mount (FIGS. 7B and 7C). The chucks can be linearly moved apart such that the solidified sample fails, which can be when the solidified sample fractures or separates (FIG. 7D). The change in length of the solidified sample, the change in transverse dimension of the solidified sample's gauge portion, and/or the force exerted on the solidified sample (e.g., the tensile force with which the sample is being pulled) (e.g., using a displacement gauge (e.g., 178), one or more cameras (e.g., 182), and/or a load cell (e.g., 186)) can be measured as the chucks are linearly moved apart to ascertain the mechanical properties of the solidified sample as set forth above.

As explained above, the solidified sample is shaped to encourage failure in the gauge portion of the solidified sample such that, across different tests of different samples, failure tends to occur at the same location to yield consistent and reliable measurements. In conventional tensile tests of a sample having a smaller-transverse-dimension gauge portion disposed between wider shoulder portions, the chucks holding the sample each comprise a grip that clamps the shoulder portions. This can pose challenges for compressible samples like clotted blood, as the clamping can induce stress and deformation in the shoulder portions of the sample that increases the risk of inadvertent sample failure in one of the shoulder portions and accordingly decrease the reliability of the measurements of the sample's mechanical properties. By forming the solidified sample's shoulder portions in portions of a mold cavity defined by the chucks such that the above-described adhesive and vacuum forces between the chucks and shoulder portions—and optionally sample branches formed in conduits of the chucks-hold the shoulder portions in the chucks, the risk of inadvertent sample failure in the shoulder portions can be mitigated to encourage failure in in the gauge portion and thereby promote the reliability of the measurements of the sample's mechanical properties.

Some of the present molds comprise two chucks and two or more central mold parts, and some of the present systems comprise a mold having two chucks and two or more central mold parts. In some embodiments, the central mold parts are configured to be removably coupled to the chucks such that the central mold parts extend between the chucks. In some embodiments, when the central mold parts are coupled to the chucks, the central mold parts and the chucks define a mold cavity. Some of the present methods of testing a sample comprise disposing the sample in a mold cavity defined by two chucks and two or more central mold parts that are removably coupled to and extend between the chucks.

The mold cavity may include a gauge-forming portion that is defined by the central mold parts and has a first transverse dimension. The first transverse dimension, in some embodiments, is between 5 and 10 millimeters (mm). The mold cavity, in some embodiments, has two shoulder-forming portions, each defined by a respective one of the chucks. Each of the shoulder-forming portions, in some embodiments, has a second transverse dimension that is at least 1.5 times the first transverse dimension. In some embodiments, the second transverse dimension is between 15 and 25 mm. In some embodiments, for each of the chucks, the shoulder-forming portion of the mold cavity defined by the chuck does not extend to an exterior of the mold when the central mold parts are coupled to the chucks.

The mold cavity, in some embodiments, may further include two expanding portions. In some embodiments, each of the expanding portions is defined by the central mold parts. Each of the expanding portions, in some embodiments, extends between the gauge-forming portion and a respective one of the shoulder-forming portions. In some embodiments, each of the expanding portions has a transverse dimension that increases moving from the gauge-forming portion to the respective shoulder-forming portion.

Each of the chucks, in some embodiments, may include a plurality of conduits. For each of the chucks, in some embodiments, the conduits of the chuck extend to an exterior of the chuck. In some embodiments, the conduits of each of the chucks chuck include a plurality of first conduits that each extend in a first direction and a plurality of second conduits that each extend in a second direction. The second direction, in some embodiments, is substantially perpendicular to the first direction. In some embodiments, for each of the chucks, the first conduits include at least ten first conduits. For each of the chucks, in some embodiments, the second conduits include at least ten second conduits. In some embodiments, for each of the chucks, the conduits of the chuck include one or more sets of conduits that each comprise two or more of the first conduits and two or more of the second conduits. For each of the set(s) of conduits, in some embodiments, the first and second conduits of the set are coplanar. In some embodiments, for each of the set(s) of conduits, each of the first conduits of the set intersects at least one of the second conduits of the set. For each of the chucks, in some embodiments, a body of the chuck includes, for each of the set(s) of conduits, a plurality of portions that are each circumscribed by two of the first conduits of the set and two of the second conduits of the set.

In some embodiments, when the central mold parts are coupled to the chucks, for each of the chucks the conduits of the chuck may be in fluid communication with the shoulder-forming portion of the mold cavity defined by the chuck. When the central mold parts are coupled to the chucks, in some embodiments, for each of the chucks, each of the conduits of the chuck extends in a direction that is substantially perpendicular to a longitudinal axis that extends through the gauge-forming portion and the shoulder-forming portions of the mold cavity. In some embodiments, each of the conduits of each of the chucks has a transverse dimension that is between 3 and 8 millimeters (mm). In some methods, disposing the sample in the mold cavity is performed such that the sample flows into the conduits of each of the chucks.

In some embodiments, each of the chucks and each of the central mold parts has one or more interior surfaces, the interior surface(s) of each of the chucks having a higher surface roughness than the interior surface(s) of each of the central mold parts. When the central mold parts are coupled to the chucks, in some embodiments, for each of the chucks, at least one of the interior surface(s) of the chuck defines the shoulder-forming portion of the mold cavity. In some embodiments, when the central mold parts are coupled to the chucks, the interior surfaces of the central mold parts define the gauge-forming portion of the mold cavity. In some embodiments, the interior surface(s) of each of the central mold parts comprise acrylic. The chucks and the central mold parts, in some embodiments, each comprise a polymeric material.

In some embodiments, the two or more central mold parts comprise one or more first central mold parts and one or more second central mold parts. In some methods, disposing the sample in the mold cavity comprises coupling the first central mold part(s) to the chucks such that the first central mold part(s) extend between the chucks. Disposing the sample in the mold cavity, in some methods, comprises placing the chucks and the first central mold part(s) in a container and dispensing the sample into the container while the chucks and the first central mold part(s) are disposed in the container and the first central mold part(s) are coupled to the chucks. In some embodiments, disposing the sample in the mold cavity comprises, after dispensing the sample into the container, coupling the second central mold part(s) to the chucks in the container such that the second central mold part(s) extend between the chucks.

Some methods comprise solidifying the sample while the sample is disposed in the mold cavity. Some of the present samples have a compressible body. The compressible body, in some embodiments, includes two shoulder portions and a gauge portion disposed between the shoulder portions. In some embodiments, for each of the shoulder portions, the sample comprises a plurality of branches coupled to the shoulder portion. The branches, in some embodiments, each extend in a direction that is substantially perpendicular to a longitudinal axis that extends through the shoulder portions and the gauge portion. The gauge portion, in some embodiments, has the first transverse dimension. Each of the shoulder portions, in some embodiments, has the second transverse dimension.

In some embodiments, for each of the shoulder portions of the sample, the branches coupled to the shoulder portion include a plurality of first branches that each extend in a first direction and a plurality of second branches that each extend in a second direction that is substantially perpendicular to the first direction. For each of the shoulder portions of the sample, in some embodiments, the first branches coupled to the shoulder portion include at least ten first branches. In some embodiments, for each of the shoulder portions of the sample, the second branches coupled to the shoulder portion include at least ten second branches. For each of the shoulder portions of the sample, in some embodiments, the branches coupled to the shoulder portion include one or more sets of branches that each comprise two or more of the first branches and two or more of the second branches. In some embodiments, for each of the set(s) of branches, the first and second branches of the set are coplanar. For each of the set(s) of branches, in some embodiments, each of the first branches of the set intersects at least one of the second branches of the set. In some embodiments, for each of the shoulder portions of the sample, for each of the set(s) of conduits, there are a plurality of openings disposed in the shoulder portion that are each circumscribed by two of the first branches of the set and two of the second branches of the set. Each of the branches of each of the shoulder portions of the sample, in some embodiments, has a transverse dimension that is between 3 and 8 mm.

In some embodiments, the compressible body further comprises two expanding portions, each extending between the gauge portion and a respective one of the shoulder portions and having a transverse dimension that increases moving from the gauge portion to the respective shoulder portion.

In some embodiments, the sample disposed in the mold cavity comprises blood and the solidified sample comprises solidified blood. In some methods, solidifying the sample comprises adding one or more clotting agents to the sample. The clotting agent(s), in some embodiments, include calcium chloride and/or thrombin. The solidified blood, in some embodiments, includes fibrin.

Some systems comprise an actuator configured to be removably coupled to a first one of the chucks. Some systems comprise a mount configured to be removably coupled to a second one of the chucks. In some systems, when the actuator is coupled to the first chuck, the mount is coupled to the second chuck, and the central mold parts are not coupled to the chucks, the actuator is configured to move the first chuck linearly relative to the second chuck along a longitudinal axis that extends through the chucks. Some methods comprise, after the sample is solidified, decoupling the central mold parts from the chucks such that a gauge portion of the solidified sample formed in the gauge-forming portion of the mold cavity is disposed between the chucks and each of two shoulder portions of the solidified sample formed in a respective one of the shoulder-forming portions of the mold cavity is disposed in a respective one of the chucks. Some methods comprise linearly moving the chucks apart along a longitudinal axis that extends through the chucks and the solidified sample. In some methods, linearly moving the chucks apart comprise coupling the first chuck to the actuator and the second chuck to the mount and moving the first chuck linearly relative to the second chuck along the longitudinal axis. The longitudinal axis, in some embodiments, is horizontal.

Some systems comprise a load cell configured to measure a force exerted on the second chuck in a direction along the longitudinal axis that extends through the chucks when the actuator is coupled to the first chuck and the mount is coupled to the second chuck. In some systems, the actuator comprises a displacement gauge. The displacement gauge, in some systems, is configured to measure a distance that the first chuck moves relative to the second chuck along the longitudinal axis that extends through the chucks when the actuator is coupled to the first chuck and the mount is coupled to the second chuck. Some systems comprise one or more cameras configured to capture video of a region between the chucks when the actuator is coupled to the first chuck and the mount is coupled to the second chuck.

Some methods comprise measuring a force exerted on the solidified sample and/or a change in length of the solidified sample when the chucks are linearly moved apart. In some embodiments, the chucks are linearly apart until failure of the solidified sample.

The term “coupled” is defined as connected, although not necessarily directly, and not necessarily mechanically; two items that are “coupled” may be unitary with each other. The terms “a” and “an” are defined as one or more unless this disclosure explicitly requires otherwise. The term “substantially” is defined as largely but not necessarily wholly what is specified—and includes what is specified; e.g., substantially 90 degrees includes 90 degrees and substantially parallel includes parallel—as understood by a person of ordinary skill in the art. As used herein, “substantially parallel” and “substantially aligned” each mean within 10 degrees of parallel to, and “substantially perpendicular” means within 10 degrees of perpendicular to.

The terms “comprise” and any form thereof such as “comprises” and “comprising,” “have” and any form thereof such as “has” and “having,” and “include” and any form thereof such as “includes” and “including” are open-ended linking verbs. As a result, an apparatus or system that “comprises,” “has,” or “includes” one or more elements possesses those one or more elements but is not limited to possessing only those elements. Likewise, a method that “comprises,” “has,” or “includes” one or more steps possesses those one or more steps but is not limited to possessing only those one or more steps.

Any embodiment of any of the apparatuses, systems, and methods can consist of or consist essentially of—rather than comprise/have/include—any of the described steps, elements, and/or features. Thus, in any of the claims, the term “consisting of” or “consisting essentially of” can be substituted for any of the open-ended linking verbs recited above in order to change the scope of a given claim from what it would otherwise be using the open-ended linking verb.

Further, an apparatus or system that is configured in a certain way is configured in at least that way, but it can also be configured in other ways than those specifically described.

The feature or features of one embodiment may be applied to other embodiments, even though not described or illustrated, unless expressly prohibited by this disclosure or the nature of the embodiments.

The above specification and examples provide a complete description of the structure and use of illustrative embodiments. Although certain embodiments have been described above with a certain degree of particularity, or with reference to one or more individual embodiments, those skilled in the art could make numerous alterations to the disclosed embodiments without departing from the scope of this invention. As such, the various illustrative embodiments of the products, systems, and methods are not intended to be limited to the particular forms disclosed. Rather, they include all modifications and alternatives falling within the scope of the claims, and embodiments other than the one shown may include some or all of the features of the depicted embodiment. For example, elements may be omitted or combined as a unitary structure, and/or connections may be substituted. Further, where appropriate, aspects of any of the examples described above may be combined with aspects of any of the other examples described to form further examples having comparable or different properties and/or functions, and addressing the same or different problems. Similarly, it will be understood that the benefits and advantages described above may relate to one embodiment or may relate to several embodiments.

The claims are not intended to include, and should not be interpreted to include, means-plus- or step-plus-function limitations, unless such a limitation is explicitly recited in a given claim using the phrase(s) “means for” or “step for,” respectively.

1. A mold comprising:
two chucks; and
two or more central mold parts that are configured to be removably coupled to the chucks such that the central mold parts extend between the chucks;
wherein when the central mold parts are coupled to the chucks, the central mold parts and the chucks define a mold cavity comprising:
a gauge-forming portion that is defined by the central mold parts and has a first transverse dimension; and
two shoulder-forming portions, each defined by a respective one of the chucks and having a second transverse dimension that is at least 1.5 times the first transverse dimension.

2. The mold of claim 1, wherein:
each of the chucks comprises a plurality of conduits; and
when the central mold parts are coupled to the chucks, for each of the chucks the conduits of the chuck:
are in fluid communication with the shoulder-forming portion of the mold cavity defined by that chuck; and
each extend in a direction that is substantially perpendicular to a longitudinal axis that extends through the gauge-forming portion and the shoulder-forming portions of the mold cavity.

3. The mold of claim 2, wherein for each of the chucks, the conduits of the chuck include:
a plurality of first conduits that each extend in a first direction; and
a plurality of second conduits that each extend in a second direction that is substantially perpendicular to the first direction.

4. The mold of claim 3, wherein for each of the chucks:
the conduits of the chuck include one or more sets of conduits that each comprise two or more of the first conduits and two or more of the second conduits; and
for each of the set(s) of conduits:
the first and second conduits of the set are coplanar; and
each of the first conduits of the set intersects at least one of the second conduits of the set.

5. The mold of claim 4, wherein for each of the chucks, a body of the chuck includes, for each of the set(s) of conduits, a plurality of portions that are each circumscribed by two of the first conduits of the set and two of the second conduits of the set.

6. The mold of claim 3, wherein for each of the chucks:
the first conduits include at least ten first conduits; and
the second conduits include at least ten second conduits.

7. The mold of claim 2, wherein each of the conduits of each of the chucks has a transverse dimension that is between 3 and 8 millimeters (mm).

8. The mold of claim 2, wherein for each of the chucks:
the conduits of the chuck extend to an exterior of the chuck; and
the respective shoulder-forming portion of the mold cavity defined by the chuck does not extend to an exterior of the mold when the central mold parts are coupled to the chucks.

9. A system for testing a sample, the system comprising:
a mold having:
two chucks; and
two or more central mold parts that are configured to be removably coupled to the chucks such that the central mold parts extend between the chucks;
wherein when the central mold parts are coupled to the chucks, the central mold parts and the chucks define a mold cavity comprising:
a gauge-forming portion that is defined by the central mold parts and has a first transverse dimension; and
two shoulder-forming portions, each defined by a respective one of the chucks and having a second transverse dimension that is at least 1.5 times the first transverse dimension;
an actuator configured to be removably coupled to a first one of the chucks; and
a mount configured to be removably coupled to a second one of the chucks;
wherein when the actuator is coupled to the first chuck, the mount is coupled to the second chuck, and the central mold parts are not coupled to the chucks, the actuator is configured to move the first chuck linearly relative to the second chuck along a longitudinal axis that extends through the chucks.

10. The system of claim 9, wherein:
each of the chucks comprises a plurality of conduits; and
when the central mold parts are coupled to the chucks, for each of the chucks the conduits of the chuck:
are in fluid communication with the shoulder-forming portion of the mold cavity defined by that chuck; and
each extend in a direction that is substantially perpendicular to a longitudinal axis that extends through the gauge-forming portion and the shoulder-forming portions of the mold cavity.

11. The system of claim 10, wherein for each of the chucks, the conduits of the chuck include:
a plurality of first conduits that each extend in a first direction; and
a plurality of second conduits that each extend in a second direction that is substantially perpendicular to the first direction.

12. The system of claim 11, wherein for each of the chucks:
the conduits of the chuck include one or more sets of conduits that each comprise two or more of the first conduits and two or more of the second conduits; and
for each of the set(s) of conduits:
the first and second conduits of the set are coplanar; and
each of the first conduits of the set intersects at least one of the second conduits of the set.

13. The system of claim 12, wherein for each of the chucks, a body of the chuck includes, for each of the set(s) of conduits, a plurality of portions that are each circumscribed by two of the first conduits of the set and two of the second conduits of the set.

14. The system of claim 11, wherein each of the conduits of each of the chucks has a transverse dimension that is between 3 and 8 millimeters (mm).

15. The system of claim 11 wherein for each of the chucks:
the first conduits include at least ten first conduits; and
the second conduits include at least ten second conduits.

16. The system of claim 10, wherein for each of the chucks:
the conduits of the chuck extend to an exterior of the chuck; and
the shoulder-forming portion of the mold cavity defined by the chuck does not extend to an exterior of the mold when the central mold parts are coupled to the chucks.

17. A sample having a compressible body that includes:
two shoulder portions;
a gauge portion disposed between the shoulder portions; and
for each of the shoulder portions, a plurality of branches coupled to the shoulder portion, each of the branches extending in a direction that is substantially perpendicular to a longitudinal axis that extends through the shoulder portions and the gauge portion;
wherein the gauge portion has a first transverse dimension and each of the shoulder portions has a second transverse dimension that is at least 1.5 times the first transverse dimension.

18. The sample of claim 17, wherein the sample comprises solidified blood that includes fibrin.

19. The sample of claim 17, wherein the compressible body further comprises two expanding portions, each:
extending between the gauge portion and a respective one of the shoulder portions; and
having a transverse dimension that increases moving from the gauge portion to the respective shoulder portion.

20. The sample of claim 17, wherein for each of the shoulder portions, the branches coupled to the shoulder portion include:
a plurality of first branches that each extend in a first direction; and
a plurality of second branches that each extend in a second direction that is substantially perpendicular to the first direction.

21. The sample of claim 20, wherein for each of the shoulder portions:
the branches coupled to the shoulder portion include one or more sets of branches that each comprise two or more of the first branches and two or more of the second branches; and
for each of the set(s) of branches:
the first and second branches of the set are coplanar; and
each of the first branches of the set intersects at least one of the second branches of the set.

22. The sample of claim 21, wherein for each of the shoulder portions, for each of the set(s) of branches, there are a plurality of openings disposed in the shoulder portion that are each circumscribed by two of the first branches of the set and two of the second branches of the set.

23. The sample of claim 20, wherein for each of the shoulder portions:
the first branches coupled to the shoulder portion include at least ten first branches; and
the second branches coupled to the shoulder portion include at least ten second branches.

24. The sample of claim 17, wherein each of the branches of each of the shoulder portions has a transverse dimension that is between 3 and 8 millimeters (mm).

25. The sample of claim 17, wherein:
the first transverse dimension is between 5 and 10 mm; and
the second transverse dimension is between 15 and 25 mm.
